(19) United States
(12) Patent Application Publication
(10) Pub. No.: US 2017/0202525 A1
(43) Pub. Date: Jul. 20, 2017

(54) METHODS AND SYSTEMS FOR ANATOMICAL STRUCTURE AND TRANSCATHETER DEVICE VISUALIZATION

(71) Applicant: THE ROYAL INSTITUTION FOR THE ADVANCEMENT OF LEARNING/MCGILL UNIVERSITY, Montreal (CA)

(72) Inventors: Nicolo PIAZZA, Dollard des Ormeaux (CA); Pascal THERIAULT-LAUZIER, Montreal (CA)

(21) Appl. No.: 15/312,821

(22) PCT Filed: May 21, 2015
(86) PCT No.: PCT/CA2015/000326

§ 371 Date: Nov. 21, 2016

Related U.S. Application Data
(60) Provisional application No. 62/001,159, filed on May 21, 2014.

(51) Int. Cl. A61B 6/12; A61F 2/24; A61B 6/00

(52) U.S. Cl. CPC A61B 6/12; A61B 6/487; A61B 6/503; A61B 6/545; A61F 2/2433; A61B 2090/3764

(57) ABSTRACT

Transcatheter aortic valve implantation (TAVI) is one of a series of catheter intervention procedures to deliver a prosthesis, in this case prosthetic valve, two structures, the aortic annular plane and the tip of the delivery catheter, must be are optimally visualized by the surgeon and in many instances there exists only one viewing angle for this. The preferred viewing angle being determined by obtaining angulation data for two views perpendicular to first and second planar structures, calculating normal vectors of each of the first and second planar structures using the angulation data, calculating a perpendicular unit vector using the normal vectors, and calculating angulation of the unit vector to establish the preferred viewing angle.

### CROSS-REFERENCE TO RELATED APPLICATIONS

This patent application claims the benefit of U.S. Provisional Patent Application 62/001,159filed May 21, 2014 entitled “Methods and Systems for Anatomical Structure and Transcatheter Device Visualization”, the entire contents of which are included by reference.

### FIELD OF THE INVENTION

This invention relates to transcatheter device implantation and more particularly to determining the view angle to be employed in transcatheter device implantation that allows both an anatomic structure and the delivery catheter to be viewed in the appropriate configuration.

### BACKGROUND OF THE INVENTION

Aortic stenosis is one of the most common valve pathologies found in adults. Aortic valve replacement via a sternotomy and cardiopulmonary bypass have been the treatment of choice for patients with symptomatic aortic stenosis with very acceptable risk. However, for patients with advanced age and multiple comorbidities this carries significant operative risk with an operative mortality as high as 25% was reported by many groups. Many of these patients are deemed nonsurgical for conventional aortic valve replacement by their cardiologists and surgeons. However, with novel surgical techniques and valve technology these patients have an alternative treatment for aortic valve stenosis. Endovascular transcatheter aortic valve replacement is one such novel surgical technique that lowers the risk in this subset of difficult patients. Furthermore, removing the need for invasive, expensive, and labour intensive techniques of sternotomy and cardiopulmonary bypass would be beneficial generally to those with aortic stenosis.

Transcatheter aortic valve implantation (TAVI) is an interventional procedure with low invasion during which the patient's diseased aortic valve is replaced by a prosthetic valve. In contrast with surgical valve replacement, during a TAVI the valve is mounted on a catheter and delivered via the patients' own vessels, thus avoiding open-heart surgery. X-ray fluoroscopy is used to visualize position the device. During the TAVI procedure the aortic root and the prosthetic valve delivery catheter should both be visualized in the optimal angular orientation. For example, planar structures, such as the aortic annular plane and the tip of the delivery catheter, are optimally visualized when they are perpendicular to the X-ray source-to-detector direction. However, for any given patient, there exists only one viewing angle that shows both the aortic root and the catheter in this optimal configuration. Adopting this view angle for implantation should lead to improved procedural outcomes.

Accordingly, it would be beneficial to determine this optimal viewing angle after having positioned the delivery catheter across the aortic root.

Within the prior art whilst several commercial software packages have been developed, such as C-THV by Paieon and 3Mensio by Pie Medical. Considering C-THV then based upon two aortograms the software presents the physician with a series of available projections from which the physician chooses their preferred working projection. The projections thus determined show the aortic root perpendicularly. In contrast 3Mensio Valves™ creates high quality three-dimensional reconstructions from X-ray computer tomography angiography, ultrasound, and angiography images. Accordingly, 3Mensio Valves™ exploits dedicated internal workflows to provide these 3D images which are geared primarily to analyzing the aortic valve and aiding the physician in the right operative approach. Fluoroscopic views that are perpendicular to the aortic root can be determined preoperatively.

However, none of the prior art software packages allow physicians to determine the appropriate viewing angle that simultaneously show two structures perpendicularly. The proposed method according to embodiments of the invention is intended to allow physicians to determine these angulations while within the fluoroscopic imaging suite.

Other aspects and features of the present invention will become apparent to those ordinarily skilled in the art upon review of the following description of specific embodiments of the invention in conjunction with the accompanying figures.

### SUMMARY OF THE INVENTION

It is an object of the present invention to address limitations within the prior art relating to transcatheter device implantation and more particularly to determining the view angle to be employed in transcatheter device implantation that allows both an anatomic structure and the delivery catheter to be viewed in the appropriate configuration.

In accordance with an embodiment of the invention there is provided a method of determining a preferred viewing angle for monitoring a transcatheter device replacement comprising:
obtaining angulation data for two views perpendicular to a first planar structure;
obtaining angulation data for two views perpendicular to a second planar structure;
calculating in dependence upon the angulation data for each of the first and second planar structures normal vectors of each of the first and second planar structures;
calculating in dependence upon the normal vectors of each of the first and second planar structures a perpendicular unit vector; and
calculating angulation of the unit vector to establish the preferred viewing angle.

In accordance with an embodiment of the invention there is provided a method of determining a preferred viewing angle for a valve replacement procedure based upon processing data obtained from computer tomography images relating to an anatomic structure and data obtained from fluoroscopy images relating to the catheter delivering the replacement valve during the procedure.

In accordance with an embodiment of the invention there is provided a non-transitory tangible computer readable medium encoding instructions for use in the execution in a computer of a method for determining a preferred viewing angle for monitoring a transcatheter device implantation in a local memory, the method comprising steps of:
obtaining angulation data for two views perpendicular to a first planar structure;
obtaining angulation data for two views perpendicular to a second planar structure;
calculating in dependence upon the angulation data for each of the first and second planar structures normal vectors of each of the first and second planar structures;
calculating in dependence upon the normal vectors of each of the first and second planar structures a perpendicular unit vector; and
calculating angulation of the unit vector to establish the preferred viewing angle.

Other aspects and features of the present invention will become apparent to those ordinarily skilled in the art upon review of the following description of specific embodiments of the invention in conjunction with the accompanying figures.

### BRIEF DESCRIPTION OF THE DRAWINGS

Embodiments of the present invention will now be described, by way of example only, with reference to the attached Figures, wherein:

FIGS. 1A and 1B 1 depicts a transcatheter aortic valve implantation procedure based upon computer aided design modeling;

FIG. 2 depicts schematically a transcatheter aortic valve implantation;

FIG. 3 depicts the typical options for insertion of a catheter to perform a transcatheter aortic valve implantation;

FIG. 4 depicts a typical catheter and a transcatheter aortic valve catheter according to the prior art;

FIGS. 5A to 5C depict the angular nomenclature employed together with images of an X-ray fluoroscopy system employed to acquire images for use by the software algorithm(s) according to embodiments of the invention;

FIG. 6 depicts the visualization as performed during a transcatheter device implantation procedure according to an embodiment of the invention;

FIG. 7 depicts the catheter visualization alignment through changing CRA/CAU angle for a RAO/LAO angle;

FIG. 8 depicts an exemplary process flow for establishing the viewing angle for a patient according to an embodiment of the invention;

FIG. 9 depicts an exemplary user interface presenting the output of a software routine for establishing the viewing angle for a patient according to an embodiment of the invention

FIG. 10 depicts fluoroscopic images of aortic root and delivery catheter as employed in embodiments of the invention;

FIG. 11 depicts fluoroscopic angulation measurements and implantation measurement depth as assessed from patient images;

FIG. 12 depicts the mean optimal projection curves for aortic valve annulus and delivery catheter tip according to an embodiment of the invention.

### DETAILED DESCRIPTION

The present invention is directed to transcatheter device implantation and more particularly to determining the view angle to be employed in transcatheter device implantation that allows both an anatomic structure and the delivery catheter to be viewed in the appropriate configuration.

The ensuing description provides exemplary embodiment(s) only, and is not intended to limit the scope, applicability or configuration of the disclosure. Rather, the ensuing description of the exemplary embodiment(s) will provide those skilled in the art with an enabling description for implementing an exemplary embodiment. It being understood that various changes may be made in the function and arrangement of elements without departing from the spirit and scope as set forth in the appended claims. Accordingly, whilst the embodiments of the invention are described and depicted with respect to a transcatheter aortic value implantation procedure it would be apparent to one skilled in the art that the methods and approaches described and discussed below may be applied to other transcatheter procedures.

A: Transcatheter Aortic Valve Implantation

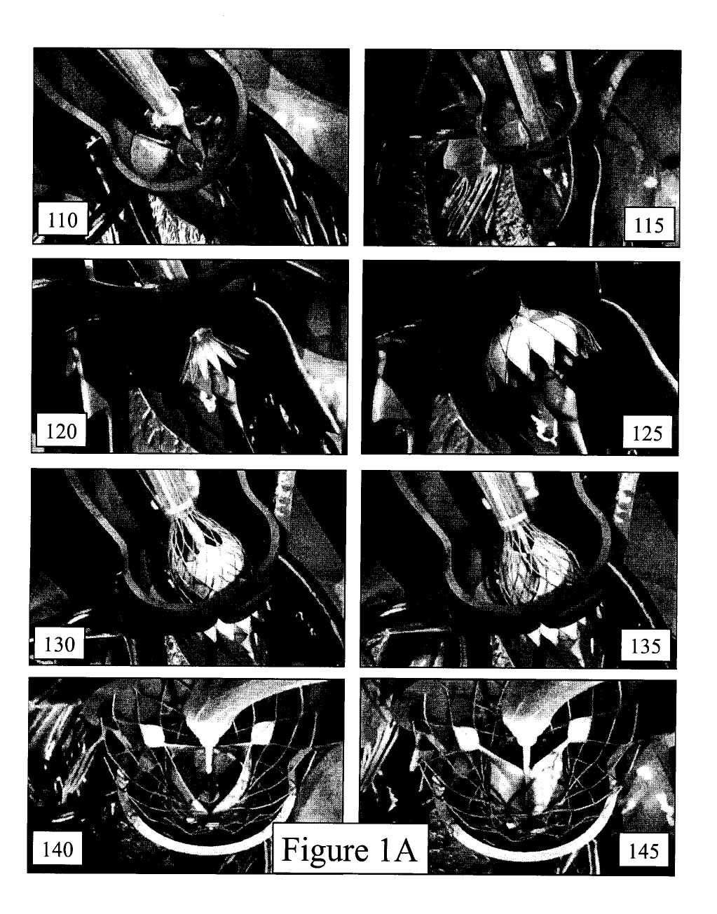

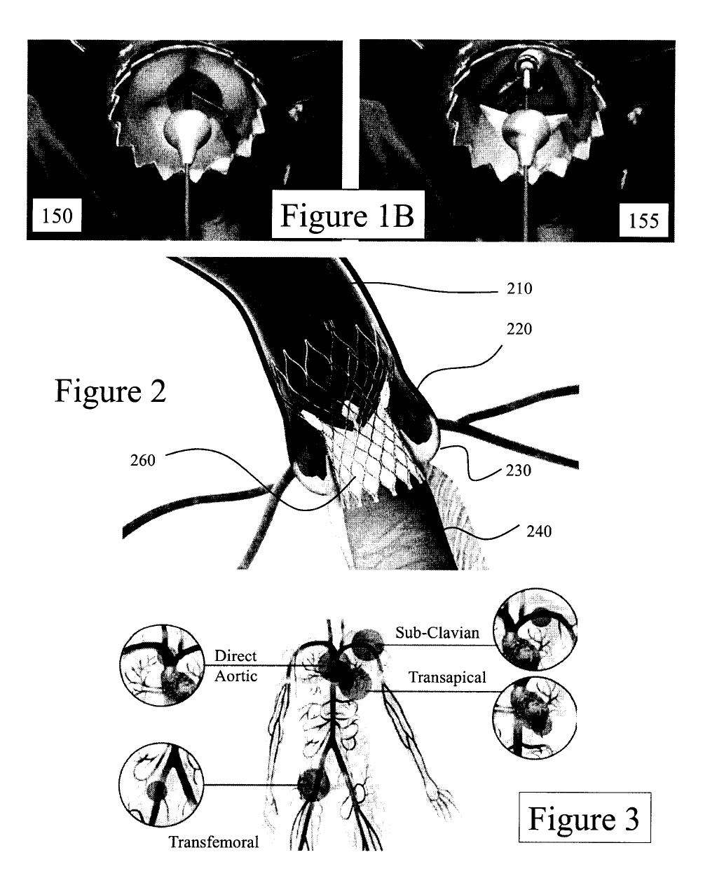

Referring to FIG. 1A and 1B there are depicted first to tenth images 110 to 155 respectively for a transcatheter aortic valve implantation procedure based upon computer aided design modeling of the deployment of a Medtronic CoreValve®. A similar system being that of SAPIEN from Edwards Lifesciences. A variety of other valves are currently undergoing development and evaluation including, but not limited to, Lotus (Boston Scientific), Direct Flow (Direct Flow Medical), HLT (Bracco), Portico (St Jude Medical), Engager (Medtronic), JenaClip (JenaValve), Acurate Valves (Symetis), and Inovare (Braile Biomedica).

As depicted in first to tenth images 110 to 155 the transcatheter aortic valve implantation procedure comprises:
Image 110 wherein the catheter has been guided to the aortic valve and section of the catheter with the replacement valve is outside the valve and heart;
Image 115 wherein the section of the catheter with the replacement valve is now positioned inside the heart on the other side of the valve;
Images 120 and 125 wherein the replacement valve deployment has been started through the catheter such that the inner annular ring of the replacement valve is released within the chamber of the heart, this being typically a skirt of polyethylene terephthalate (PET);
Images 130 and 135 wherein the deployment process continues such that the outer annular ring of retaining stainless steel metallic elements (frame) are being deployed, wherein these expand through use of a balloon to engage the inner wall of the aorta;
Images 140 and 145 wherein the deployment process continues such that the outer annular ring of retaining metallic elements are completely deployed and the leaflets of the valve are released, these being for example bovine pericardial tissue affixed to the frame and in some instances these leaflets are treated to reduce subsequent calcification during use (the valve being open in image 140 and image 145 and being comprised of three leaflets);
Images 150 and 155 show the deployed aortic valve replacement from below (i.e. within the heart chamber) in closed and open positions respectively prior to the withdrawal of the catheter.

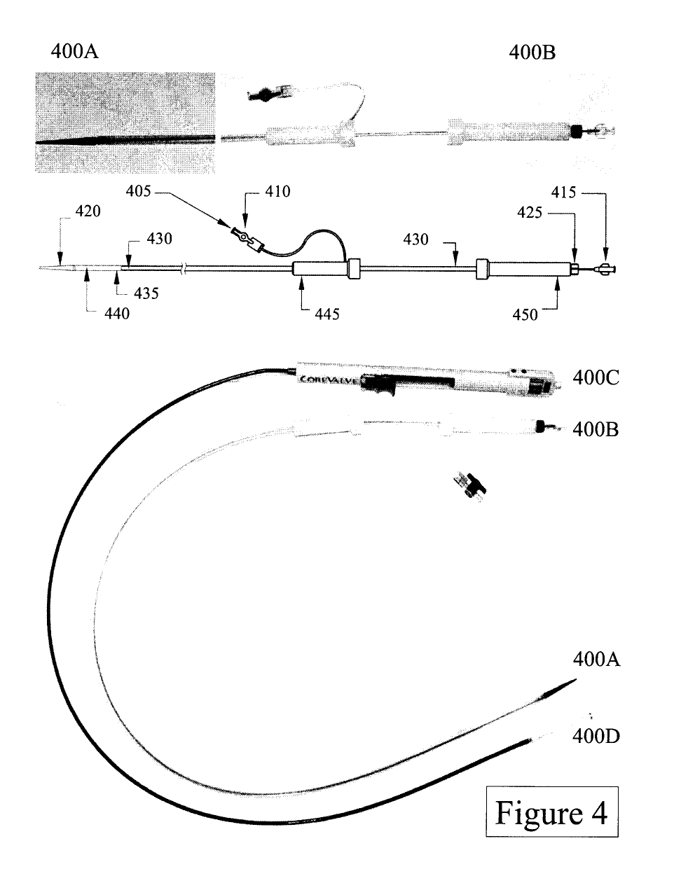

Now referring to FIG. 2 there is depicted a deployment of an aortic valve replacement 260. As depicted the aortic valve replacement 260 is positioned at the valve between the ascending aorta 210 and left ventricle 240 of the patient's heart. Also depicted are the aortic sinuses 220 with their coronary ostia and aortic valve annulus 230. Deployment of the transcatheter aortic valve replacement 260 may be achieved through the catheter being introduced into the patient's blood vessels and directed to their heart. The most common catheter insertion points are depicted in FIG. 3 and are direct aortic, transfemoral, transapical, and sub-clavian. Referring to FIG. 4 there are depicted conventional a conventional catheter comprising first deployment end 400A and manipulation end 400B and a CoreValve™ catheter with second manipulation end 400C and second deployment end 400D. Whilst the conventional and CoreValve™ catheters differ in the design of the deployment ends their functionalities are basically the same in that through manipulation of the manipulation ends the user may execute the sequential stages of deployment as described supra in respect of FIGS. 1A and 1B. Considering the conventional catheter then this comprises:

Flush port 405;

One-way valve 410;

Guidewire hub 415;

Atraumatic tip 420;

Haemostasis valve 425;

Stabilizer tube 430;

Outer shaft 435;

Valve loading space 440;

Deployment handle 445; and

Stablizer handle 450.

Accordingly, based upon the length of the stabilizer tube 430 the catheter may be used in the different deployment scenarios described supra in respect of FIG. 3.

B. Fluoroscopic Imaging

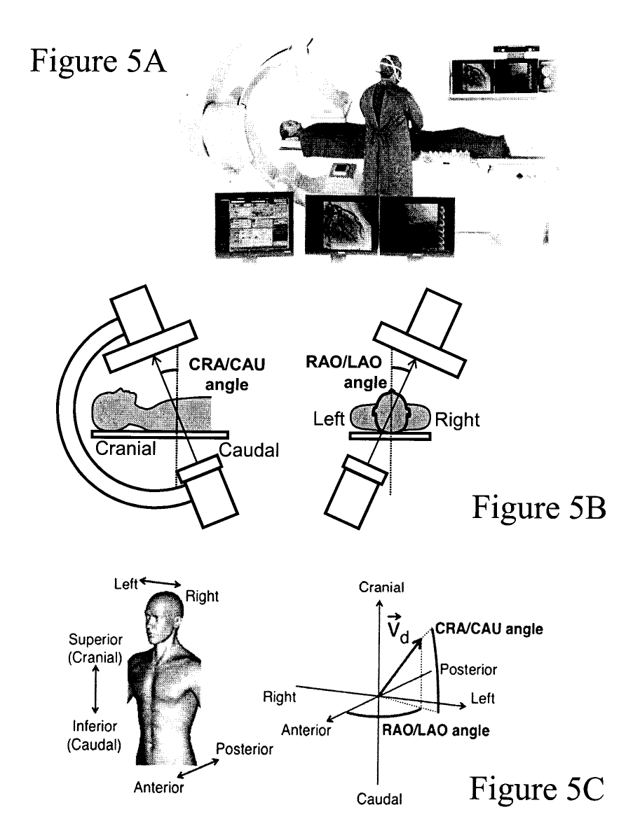

Now referring to FIG. 5A there is depicted an example of a fluoroscopic imaging system exploited in imaging in procedures such as transcatheter aortic valve implantation procedures which are subject of embodiments of the invention. Fluoroscopy is an imaging technique that uses X-rays to obtain real-time moving images of the internal structures of a patient through the use of a fluoroscope. In its simplest form, a fluoroscope consists of an X-ray source and fluorescent screen between which the patient is placed. Typically, fluoroscopes exploit an X-ray image intensifier and CCD video camera in order to allow the images to be recorded and displayed on a monitor. Due to the use of X-rays, a form of ionizing radiation, there are potential risks from the imaging procedure itself as whilst physicians try to use low dose rates during fluoroscopic procedures, the length of a typical procedure often results in a relatively high absorbed dose to the patient. Accordingly, anything that can reduce the length of the procedure and dose to the patient is beneficial above and beyond increasing the successful outcomes of the transcatheter aortic valve implantation procedures themselves.

Fluoroscopic view orientations are described using two angles, as depicted in FIG. 5B, which are the cranio-caudal angle (CRA/CAU) and a right-left anterior oblique angle (RAO/LAO). As evident from FIG. 5C CRA/CAU angles define whether the viewing is towards the upper torso, defined as superior/cranial, or the lower torso, defined as inferior/caudal. The RAO/LAO angle defines the view as being to the left or right hand sides of the patient. The combination of the CRA/CAU angle and RAO/LAO angle define a vector \vec{V}_{d} for the viewing.

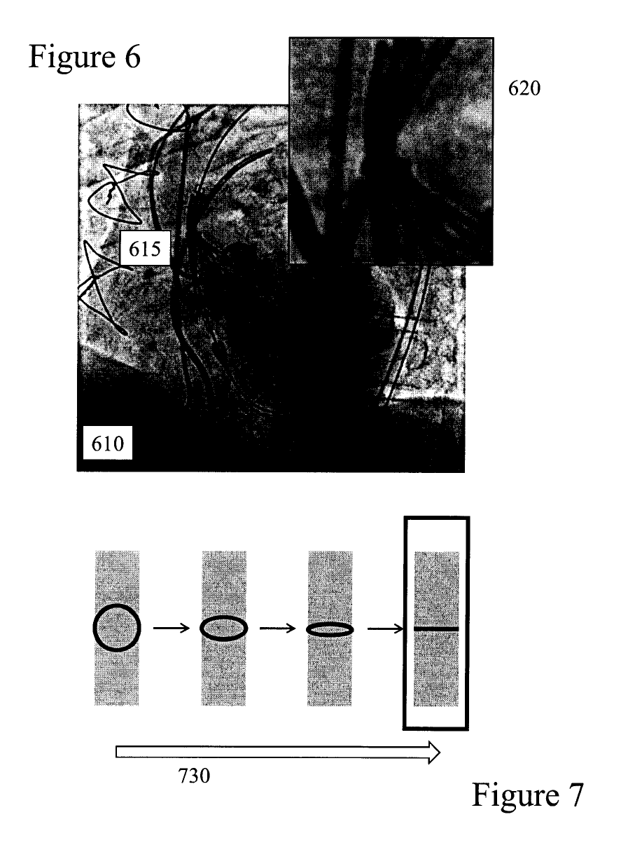

Referring to FIG. 6 there is depicted a fluoroscopy image 610 for a patient together with region 615 around the replacement aortic valve which is clearly visualized from its metallic elements and depicted in zoomed image 620. As described supra the prior art exploits computer tomography scans to define the orientation of the aortic root or the anatomical structure of interest. However, the inventors then during the operation with the catheter deployed performing additional determinations to establish the optimum angle for both visualizing the anatomy and the device. Accordingly, considering the vector \vec{V}_{d} then for a particular RAO/LAO angle there will be a CRA/CAU angle, which as it is varied, a catheter marker (e.g. a metallic band) will be seen as a line as depicted in FIG. 7. Repeating this for different RAO/LAO angles yields multiple CRA/CAU angles.

C. Fluoroscopic Angulation Algorithm

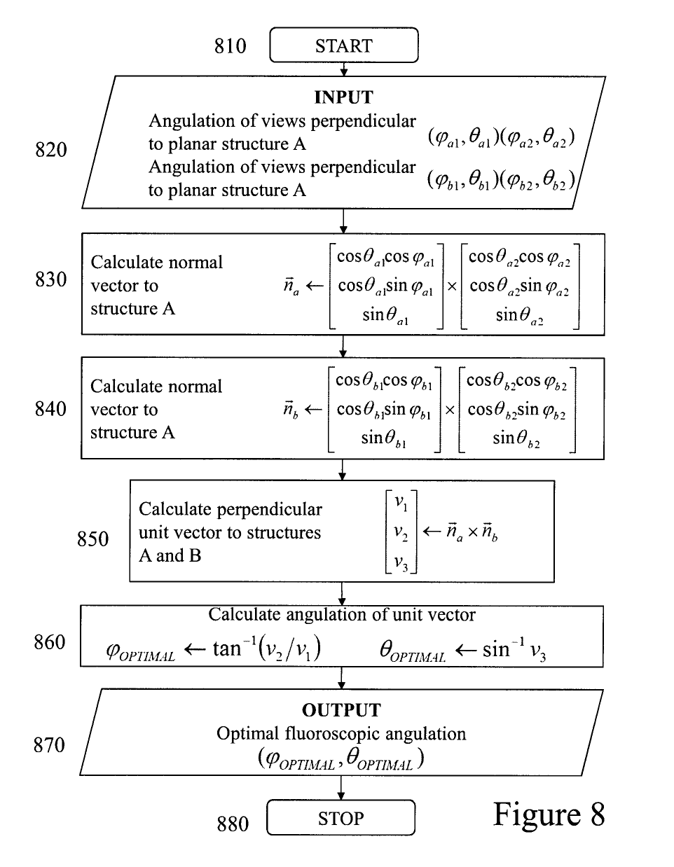

Repeating the process presented supra yields 4 values for the anatomic structure, for example derived from computer tomography scans, and 4 values for the catheter, for example derived from fluoroscopy measurements during the procedure. These values as depicted in FIG. 8 are employed within a process flow that yields two angles, these being the optimum angles for viewing both the anatomical structure and the catheter.

Accordingly, considering \vec{V}_{d} which describes the source-to-detector orientation then this may be defined by Equation (1) where θ is the CRA/CAU angle and φ is the RAO/LAO angle. For a particular planar structure, one can determine angulations of two different views that show the structure of interest perpendicularly, namely \vec{V}_{d1}(θ₁,φ₁) and \vec{V}_{d2}(θ₂,φ₂). The normal vector \vec{n} of the planar structure may be obtained using a cross-product orientation as depicted in Equations (2A) and (2B).

$$\begin{matrix}{\vec{V}}_{d} ( θ , ϕ ) = [ \begin{matrix}cos θ \cdot cos ϕ \\ cos θ \cdot sin ϕ \\ sin θ\end{matrix} ] & ( 1 ) \\ \vec{n} = {\vec{V}}_{d 1} \times {\vec{V}}_{d 2} & ( 2 A ) \\ \vec{n} = [ \begin{matrix}cos {θ}_{1} \cdot cos {ϕ}_{1} \\ cos {θ}_{1} \cdot sin {ϕ}_{1} \\ sin {θ}_{1}\end{matrix} ] \times [ \begin{matrix}cos {θ}_{2} \cdot cos {ϕ}_{2} \\ cos {θ}_{2} \cdot sin {ϕ}_{2} \\ sin {θ}_{2}\end{matrix} ] & ( 2 B )\end{matrix}$$

If two planar structures of interest exist, each with its own normal vector \vec{n}ₐ and \vec{n}_{b}, then one can determine an optimal direction, i.e. the direction that is perpendicular to each structure, using again a cross-product operation as described in Equation (3), where \vec{V}_{OPTIMAL} is the unit vector describing the optimal direction. Subsequently, one can determine the fluoroscopic angulation corresponding to the optimal direction as defined by Equation (4) as determined using Equations (5A) and (5B).

$$\begin{matrix}{\vec{V}}_{OPTIMAL} = {\vec{n}}_{a} \times {\vec{n}}_{b} & ( 3 ) \\ {\vec{V}}_{OPTIMAL} = [ \begin{matrix}{v}_{1} \\ {v}_{2} \\ {v}_{3}\end{matrix} ] & ( 4 ) \\ {θ}_{OPTIMAL} = {sin}^{- 1} ( {v}_{2} ) & ( 5 A ) \\ {ϕ}_{OPTIMAL} = {tan}^{- 1} ( \frac{{v}_{2}}{{v}_{1}} ) & ( 5 B )\end{matrix}$$

Accordingly, the algorithm depicted in respect of FIG. 8 takes as input eight angles from four fluoroscopic views. Accordingly, in step 810 the process starts and in step 820 captures angulation of views that are perpendicular to planar structure A, namely (θ_{A1},φ_{A1}) and (θ_{A2},φ_{A2}), as well as angulation of views that are perpendicular to planar structure B, namely (θ_{B1},φ_{B1}) and (θ_{B2},φ_{B2}). Next in step 830 the process calculates the normal vector to structure A as given by Equation (6) before calculating the normal vector to structure B as given by Equation (7) in step 840.

$$\begin{matrix}{\vec{n}}_{A} = [ \begin{matrix}cos {θ}_{A 1} \cdot cos {ϕ}_{A 1} \\ cos {θ}_{A 1} \cdot sin {ϕ}_{A 1} \\ sin {θ}_{A 1}\end{matrix} ] \times [ \begin{matrix}cos {θ}_{A 2} \cdot cos {ϕ}_{A 2} \\ cos {θ}_{A 2} \cdot sin {ϕ}_{A 2} \\ sin {θ}_{A 2}\end{matrix} ] & ( 6 ) \\ {\vec{n}}_{B} = [ \begin{matrix}cos {θ}_{B 1} \cdot cos {ϕ}_{B 1} \\ cos {θ}_{B 1} \cdot sin {ϕ}_{B 1} \\ sin {θ}_{B 1}\end{matrix} ] \times [ \begin{matrix}cos {θ}_{B 2} \cdot cos {ϕ}_{B 2} \\ cos {θ}_{B 2} \cdot sin {ϕ}_{B 2} \\ sin {θ}_{B 2}\end{matrix} ] & ( 7 )\end{matrix}$$

Subsequently, in step 850 the perpendicular unit vector to the structure A and B is determined as given by Equation (8) from which in step 860 the angulation of the unit vector is determined as given by Equations (9A) and (9B) thereby yielding the optimal fluoroscopic angulation in step 870 before the process stops in step 880.

$$\begin{matrix}[ \begin{matrix}{v}_{1} \\ {v}_{2} \\ {v}_{3}\end{matrix} ] ← {\vec{n}}_{A} \times {\vec{n}}_{B} & ( 8 ) \\ {θ}_{OPTIMAL} ← {sin}^{- 1} ( {v}_{3} ) & ( 9 A ) \\ {ϕ}_{OPTIMAL} ← {tan}^{- 1} ( \frac{{v}_{2}}{{v}_{1}} ) & ( 9 B )\end{matrix}$$

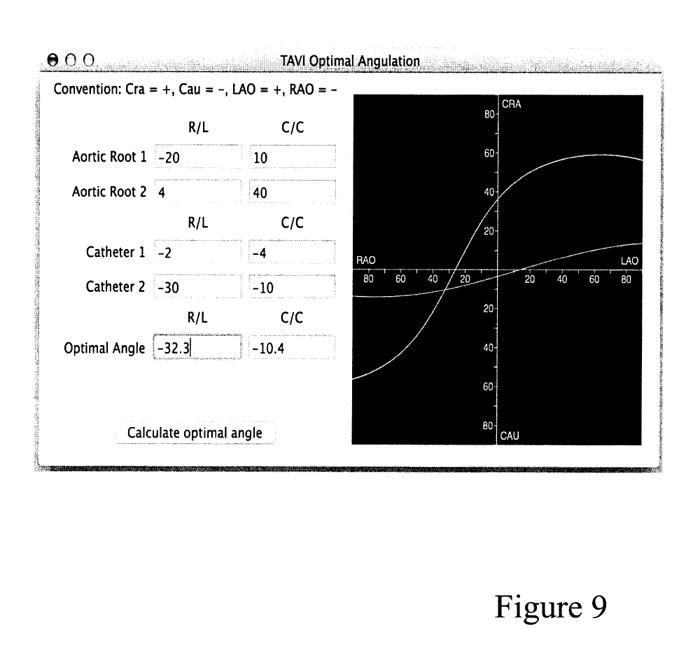

Referring to FIG. 9 there is depicted an exemplary user interface according to an embodiment of the invention exploiting the process described in respect of FIG. 8. First, a user measures the angles that allow perpendicular visualization of two structures of interest. In this case the structures of interest are labeled “Aortic Root” and “Catheter”. On each row a different fluoroscopic angulation is entered. The column labeled “R/L” corresponds to the angle φ and the column “C/C” corresponds to the angle θ. The user, then clicks “Calculate optimal angle” wherein the optimal angulation is calculated and displayed in the row labeled “Optimal Angle”.

The plot to the rightmost half of the window displays the CRA/CAU angle as a function of the RAO/LAO angle. It shows two curves, one for each of the structures of interest. The points making up each curve correspond to fluoroscopic views that are perpendicular to the structure of interest. Therefore, the intersection point of both curves represents the optimal angle that shows both structures of interest simultaneously in a perpendicular orientation.

D. Fluoroscopic Angulation Procedure

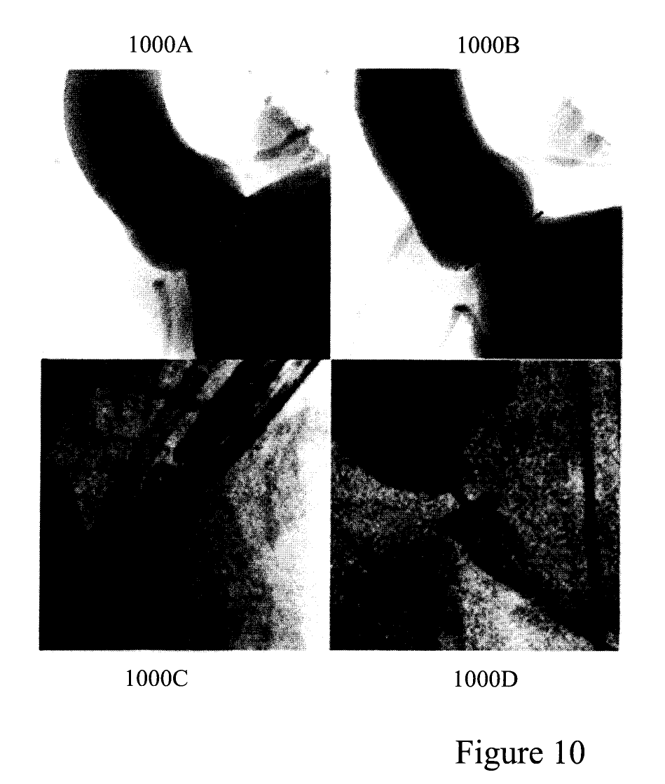

During a TAVR, the aortic root and the prosthetic valve delivery catheter should both be visualized in an optimal angulation, such as depicted in FIG. 10. Planar structures, such as at the aortic annular plane and the tip of the delivery catheter, are optimally visualized when they are perpendicular to the source-to-detector direction, i.e. when they are coplanar. For any given patient, there exists only one view angle that shows both the aortic root and the catheter in an optimal configuration. The proposed method allows one to determine this optimal viewing angle after having positioned the delivery catheter across the aortic root. To our knowledge, this is the first method to achieve this optimal viewing angle.

As noted above, two angles are typically used to describe C-arm fluoroscopic angulations, the cranial/caudal (CRA/CAU) and left-anterior-oblique/right-anterior-oblique (LAO/RAO). During a TAVR, the source-to-detector direction should be orthogonal to the normal vector of the aortic valve annular plane in order to maximize positioning accuracy. Based on this criterion, it is possible to determine an optimal CRA/CAU angle for any given LAO/RAO angle. The plot of the optimal combinations is called the aortic valve optimal projection curve (OPC). This function is given by Equation (10) where φ is the cranio-caudal angle of the OPC at RAO/LAO angle θ, φ_{EN}_{_}FACE and θ_{EN}_{_}FACE are respectively the cranio-caudal and RAO/LAO angles of the aortic valve viewed en face.

$$φ = - arctan [ \frac{cos ( θ - {θ}_{EN \_ FACE} )}{tan {φ}_{EN \_ FACE}} ] \qquad ( 10 )$$

An important point to note is that the OPC can be generalized for any planar structure. Therefore, one can obtain an OPC for other anatomic structures, such as the mitral valve annulus, the os of the left atrial appendage, or the inter-atrial septum. An OPC can also be defined for implanted structures; we are particularly interested in the OPC of the delivery catheter tip. The intersection point between the OPC of two distinct structures defines a unique view angle that shows both structures optimally. Therefore, the intersection point of the OPC of the aortic valve annulus and of the TAVR delivery catheter tip defines a simultaneously optimal delivery angle for both structures.

Referring to FIG. 10 there are depicted first to fourth images 1000A to 1000D respectively which show respectively:
First image 1000A—view of the aortic root in non-coplanar angulation;
Second image 1000B—view of the aortic root in coplanar angulation;
Third image 1000C—view of the TAVR device delivery catheter in non-coplanar angulation;
Fourth image 1000D—view of the TAVR device delivery catheter in coplanar angulation.

Importantly, the fluoroscopic angulation that shows a structure en face, and thus defines the OPC, can be determined from two angulations that show the structure perpendicularly. For the aortic root, a pre-operative computed tomography (CT) scan of the patient is used to find two such angulations. Because the delivery catheter is not yet in position at the time of the pre-operative CT scan, its orientation must be determined intra-operatively. This is accomplished using simple C-arm manipulations. For a fixed LAO/RAO angle, the CRA/CAU angle is changed until the metal band at the catheter tip is seen as a line (FIG. 10). The angulation is noted and this process is repeated for a different LAO/RAO angle. The resulting angles are entered into the optimization algorithm as discussed supra in respect of FIG. 8. Note that this procedure can be applied within a few seconds and without injection of iodinated contrast agent. Furthermore, it does not require hardware or software modifications of the fluoroscopic suite.

E. Experimental Verification

E.1 Study Design

A single-arm non-randomized study to evaluate the feasibility of obtaining simultaneously coplanar fluoroscopic angulation for the aortic annulus and the TAVR delivery catheter was established with the approval of The Research Ethics Office at McGill University. The primary outcome for the study was the achievement of feasible, simultaneous coplanar angulation. This angulation was defined as a view angle that the operators were able to obtain using the fluoroscopic C-arm system used in the study and that shows both the aortic valve annulus and the delivery catheter tip in a coplanar configuration. Operators made the determination intra-operatively. Secondary desired outcomes were directed to the angulation of the coplanar configuration, the depth of implantation of the TAVR prosthesis, and the angle between the planes of the aortic annulus and the delivery catheter tip.

The fluoroscopic angulation of the coplanar configuration was obtained using the method described above. The implantation depth was defined as the distance of protrusion of the prosthesis below the aortic annulus measured between the aortic valve annulus and the prosthesis inflow end. This distance was measured on post-implantation fluoroscopic images using an imaging workstation which was calibrated for magnification using a manufacturer-provided length of the implant strut. The angle between the planes of the aortic annulus and the delivery catheter tip were calculated using the arccosine of normal vectors dot product. The normal vector was calculated from the normalized cross product of spherical coordinate unit vector from the two orthogonal fluoroscopic angulations measured for each structure. The angles were calculated using MATLAB version R2013a.

E2. Data Acquisition

A contrast enhanced CT scan was obtained for each patient using a 64-slice Discovery CT750 HD system. A proprietary prosthesis of size 23 mm, 26 mm, 29 mm, or 31 mm was selected based on CT measurements performed using Osirix™ MD image processing software. Double-oblique multi-planar reconstructions of the CT scan were also analyzed using the software package FluoroCT™ CT scan visualization software tool to determine two fluoroscopic angulations perpendicular to the aortic root. Angulations showing the delivery catheter perpendicularly were determined intra-operatively. The resulting angles were entered into the algorithm discussed supra. A Toshiba™ INFX series interventional C-arm system was used in conjunction with a digital flat panel detector.

E3. Statistical Analysis

Continuous variables were expressed as mean ±standard deviation, and categorical variables were reported as frequencies. Viewing angles are expressed as mean and 95% confidence interval. The statistical analysis was performed using MATLAB assuming that the directional data were distributed according to the von Mises-Fisher distribution. The threshold for statistical significance was set at p=0.05.

E4, Results

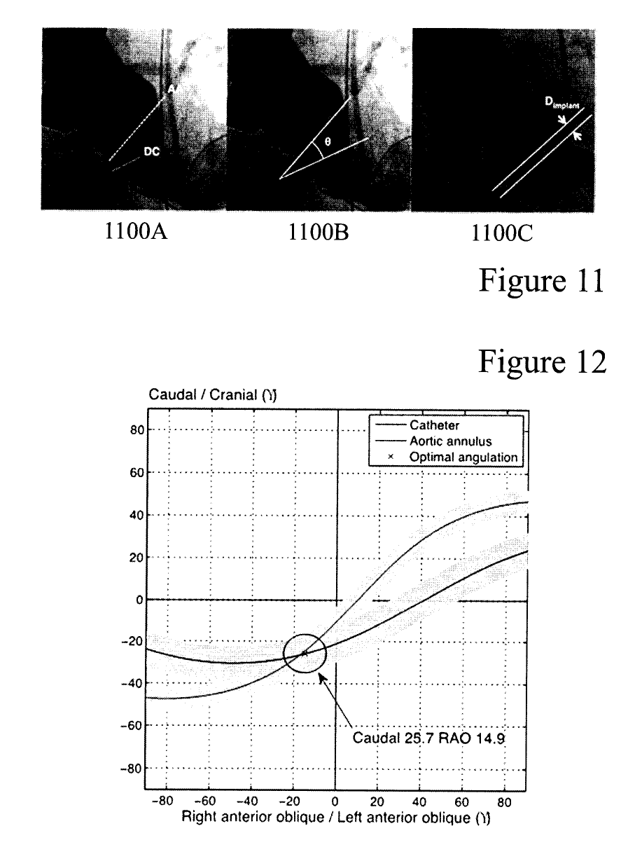

The baseline characteristics of the study population are presented in Table 1. A case example is shown in FIG. 11 and demonstrates a fluoroscopic image of the aortic root and delivery catheter immediately prior to the deployment of the prosthesis and after the application of the optimization algorithm. First image 1100A depicts the fluoroscopic angulation with simultaneously coplanar aortic valve annulus (AA) and delivery catheter tip (DC). Second image 1100B depicts the angle between aortic valve annulus and delivery catheter tip (θ) whilst third image 1100C depicts the depth of implantation (D_{IMPLANT}).

TABLE 1
Baseline Characteristics of Study Population (n = 25)

| | |
|---|---|
| Age, years | 83.6 ± 6.7 |
| Female, n | 8 (36.4%) |
| Height, m | 1.6 ± 0.1 |
| Weight, kg | 71.9 ± 14.9 |
| Body Mass Index (BMI), kg/m² | 26.9 ± 4.3 |
| Body Surface Area (BSA), m² | 1.8 ± 0.2 |
| Creatinine, μmol/L | 113.3 ± 102.9 |
| Creatinine, mg/dL | 1.3 ± 1.2 |
| Left Ventricular Ejection Fraction (LVEF), % | 55.0 ± 14.2 |
| Hypertension, n | 17 (77.3%) |
| Diabetes mellitus, n | 5 (22.7%) |
| New York Heart Association score, n | |
| I | 0 (0.0%) |
| II | 13 (59.1%) |
| III | 8 (36.4%) |
| IV | 1 (4.5%) |
| Society of Thoracic Surgeons (STS) mortality risk, % | 6.2 ± 2.1 |
| STS mortality and morbidity risk, % | 28.7 ± 7.1 |

The results for the primary and secondary outcomes are summarized in Table 2. Out of 25 patients, 24 cases resulted in a feasible fluoroscopic view angle. In one case, the view angle was RAO 87.5° CAU 48°, which lies outside the feasible range.

TABLE 2
Results for primary and secondary outcomes

| | |
|---|---|
| Cases with feasible coplanar fluoroscopic | 24 (96%) |
| angulation, n | |
| Mean coplanar fluoroscopic angulation, ° (95% CI) | |
| Right Anterior Oblique | 14.9 (4.8-25) |
| Caudal | 25.0 (16.6-34.8) |
| Mean implantation depth, mm ± SD | 3.2 ± 1.4 |
| Mean angle between aortic annulus and | 28.9 ± 11.1 |
| catheter plane, ° ± SD | |

Note:
n: number of patients,
95% CI: 95% confidence interval,
SD: standard deviation

The mean optimal projection curves for the aortic root and delivery catheter are presented in FIG. 12 with 95% confidence regions. The intersection point of both curves is the optimal implantation view angle: RAO 14.9° (95% confidence interval: RAO 4.8° to 25.0°) and CAU 25.7° (95% confidence interval: CAU 16.6° to)34.8°).

The implantation depth averaged 3.2±1.4 mm in the 25 cases. Furthermore, the mean deviation angle between the catheter and aortic valve annulus was 28.9±11.1° with a range of 5.8° to 49.0°. The difference in orientation is highly statistically significant with p=8×10⁻⁸.

E5. Discussion

The results presented supra demonstrate the feasibility of an embodiment of the invention wherein fluoroscopic angulation minimizes parallax error for both the aortic valve annulus and the TAVR delivery catheter. Prior studies have focused on the optimization of the visualization of the aortic valve alone. Within the prior art it has been demonstrated that adopting an optimal fluoroscopic angulation for the aortic valve annulus can significantly decrease implantation time, radiation exposure, the amount of injected iodinated contrast agent, the risk of acute kidney injury as well as the combined rate of valve malposition and aortic regurgitation. Given that the rate of paravalvular aortic regurgitation post-implantation is strongly associated with TAVR mortality, it can be hypothesized that optimizing the fluoroscopic angulation of the aortic valve may lead to improved outcomes in TAVR.

The depth of implantation is associated with the development of new conduction disturbance after TAVR. Within the prior art patients with a low implantation of a balloon-expandable TAVR device have been associated with clinically significant new conduction disturbance such as left bundle branch blocks and complete heart blocks; a low implantation was also correlated with a higher rate of new pacemaker implantation. In that study, patients with new conduction disturbances had an implantation depth of 5.5±2.9 mm versus 3.4±2.0 mm in patients without new conduction disturbances. In the current study, we demonstrated an average implantation depth of 3.2±1.4 mm, which leads to the hypothesis that simultaneous optimization of the fluoroscopic angulation may reduce the rate of new conduction disturbances and new pacemaker implantation after TAVR.

A large amount of inter-subject variability was observed in the optimal angulation, which provides evidence that a standard implantation view angle is unlikely to be applied to all patients. This supports that claim that optimization procedure demonstrated in this article should be applied to each case individually. Furthermore, the angle between the aortic valve annulus and the delivery catheter, averaging 28.9±11.1°, demonstrates that these two structures are never mutually coaxial. This observation is a requirement for the applicability of the proposed method. Indeed, should the aortic valve annulus and the delivery catheter be coaxial, these structures would have identical OPC. Consequently, any fluoroscopic view angle lying on the OPC would be mutually optimal for both structures, thus obviating the need for the optimization method. We thus conclude that the application of the proposed method is feasible in a majority of patients undergoing TAVR for moderate to severe symptomatic aortic regurgitation.

While the study focused on TAVR, the proposed methods can be applied to most transcatheter procedures where a device is deployed within an approximately circular or cylindrical anatomical feature. Accordingly, other applications of embodiments of the invention may include, but not be limited to, transcatheter mitral valve replacement, left atrial appendage occlusion, and atrial or ventricular septal defect occlusion.

Specific details are given in the above description to provide a thorough understanding of the embodiments. However, it is understood that the embodiments may be practiced without these specific details. For example, circuits may be shown in block diagrams in order not to obscure the embodiments in unnecessary detail. In other instances, well-known circuits, processes, algorithms, structures, and techniques may be shown without unnecessary detail in order to avoid obscuring the embodiments.

The foregoing disclosure of the exemplary embodiments of the present invention has been presented for purposes of illustration and description. It is not intended to be exhaustive or to limit the invention to the precise forms disclosed. Many variations and modifications of the embodiments described herein will be apparent to one of ordinary skill in the art in light of the above disclosure. The scope of the invention is to be defined only by the claims appended hereto, and by their equivalents.

Further, in describing representative embodiments of the present invention, the specification may have presented the method and/or process of the present invention as a particular sequence of steps. However, to the extent that the method or process does not rely on the particular order of steps set forth herein, the method or process should not be limited to the particular sequence of steps described. As one of ordinary skill in the art would appreciate, other sequences of steps may be possible. Therefore, the particular order of the steps set forth in the specification should not be construed as limitations on the claims. In addition, the claims directed to the method and/or process of the present invention should not be limited to the performance of their steps in the order written, and one skilled in the art can readily appreciate that the sequences may be varied and still remain within the spirit and scope of the present invention.

What is claimed is:

1. A method of determining a preferred viewing angle for monitoring a transcatheter device implantation comprising:
obtaining angulation data for two views perpendicular to a first planar structure;
obtaining angulation data for two views perpendicular to a second planar structure;
calculating in dependence upon the angulation data for each of the first and second planar structures normal vectors of each of the first and second planar structures;
calculating in dependence upon the normal vectors of each of the first and second planar structures a perpendicular unit vector; and
calculating angulation of the unit vector to establish the preferred viewing angle.

2. The method according to claim 1, wherein the preferred viewing angle is defined by a cranio-caudal angle (CRA/CAU) and a right-left anterior oblique angle (RAO/LAO).

3. The method according to claim 1, wherein the angulation data for two views perpendicular to the first planar structure are obtained using computer tomography images.

4. The method according to claim 1, wherein the angulation data for two views perpendicular to the second planar structure are obtained using fluoroscopy images during the valve replacement procedure.

5. A method of determining a preferred viewing angle for a valve replacement procedure based upon processing data obtained from computer tomography images relating to the anatomic structure and data obtained from fluoroscopy images relating to the catheter delivering the replacement valve during the procedure.

6. A non-transitory tangible computer readable medium encoding instructions for use in the execution in a computer of a method for determining a preferred viewing angle for monitoring a transcatheter device implantation in a local memory, the method comprising steps of:
obtaining angulation data for two views perpendicular to a first planar structure;
obtaining angulation data for two views perpendicular to a second planar structure;
calculating in dependence upon the angulation data for each of the first and second planar structures normal vectors of each of the first and second planar structures;
calculating in dependence upon the normal vectors of each of the first and second planar structures a perpendicular unit vector; and
calculating angulation of the unit vector to establish the preferred viewing angle.

7. The non-transitory tangible computer readable medium according to claim 6, wherein the preferred viewing angle is defined by a cranio-caudal angle (CRA/CAU) and a right-left anterior oblique angle (RAO/LAO).

8. The non-transitory tangible computer readable medium according to claim 6, wherein the angulation data for two views perpendicular to the first planar structure are obtained using computer tomography images.

9. The non-transitory tangible computer readable medium according to claim 6, wherein the angulation data for two views perpendicular to the second planar structure are obtained using fluoroscopy images during the valve replacement procedure.
